(19) United States
(12) Patent Application Publication
(10) Pub. No.: US 2022/0039639 A1
(43) Pub. Date: Feb. 10, 2022

(54) METHODS AND DEVICES FOR CALCULATING A LEVEL OF "CLINICAL RELEVANCE" FOR ABNORMAL SMALL BOWEL FINDINGS CAPTURED BY CAPSULE ENDOSCOPY VIDEO

(71) Applicants: ASSISTANCE PUBLIQUE-HOPITAUX DE PARIS, Paris (FR); CENTRE NATIONAL DE LA RECHERCHE SCIENTIFIQUE, Paris (FR); SORBONNE UNIVERSITÉ, Paris (FR); ÉCOLE NATIONALE SUPÉRIEURE DE L'ÉLECTRONIQUE ET DE SES APPLICATIONS, Cergy (FR); CY CERGY PARIS UNIVERSITÉ, Cergy (FR)

(72) Inventors: Xavier DRAY, Paris (FR); Romain LEENHARDT, Paris (FR); Aymeric HISTACE, Menucourt (FR)

(21) Appl. No.: 16/987,301

(22) Filed: Aug. 6, 2020

(51) Int. Cl. A61B 1/04; A61B 1/273; A61B 1/00; G06K 9/00; G16H 50/30

(52) U.S. Cl. CPC A61B 1/041; A61B 1/273; G16H 50/30; G06K 9/00718; A61B 1/00045

(57) ABSTRACT

The invention relates a method (100) and a device (200) for analyzing a video sequence captured by Small Bowel (SB) Capsule Endoscopy (CE) devices, when placed in a patient's body during a SBCE video examination, the clinical setting of the patient being chosen from overt Obscure GastroIntestinal Bleeding (OGIB), occult OGIB, or suspected Crohn's Diseases (CD), by calculating a level of “clinical relevance” of the findings detected by the SBCE devices according to the chosen clinical setting, comprising:
(a) collecting (110), by a memory, at least one video sequence captured during a SBCE video examination;
(b) automatically detecting (120), by a processor, from the video sequence, at least one image comprising at least one abnormal finding in the Small Bowel detected among sixteen types of SB findings;
(c) counting (130), by the processor, the abnormal detected findings;
(d) classifying (140), by the processor the abnormal detected findings according to predetermined rules; and
(e) outputting (150), by a display, the “clinical relevance” of each abnormal detected finding.

### TECHNICAL FIELD

This invention relates to the assessment of the clinical relevance of findings in small bowel capsule endoscopy devices and, more particularly, to methods and devices for analyzing a video sequence captured by small bowel capsule endoscopy devices.

### BACKGROUND ART

Capsule endoscopy (CE) is of great help and recommended investigating the small bowel (SB) in patients with obscure gastrointestinal bleeding (OGIB) or with suspected Crohn's disease, when gastroscopy and ileocolonoscopy are normal (1,2).

Diagnostic and prognostic evaluations are based on the reader's expertise as lesions found during the examination are usually not sampled by endoscopy or surgery for pathological assessment.

Regarding diagnosis, the European Society for Gastrointestinal Endoscopy (ESGE) SB working group has recently established consensual names and descriptions of the most frequent pathological findings seen in SBCE (3,4), in order to better standardize CE reading and teaching, and to guide research study protocols.

Regarding prognosis, although misinterpretation of the clinical relevance of lesions can lead to inappropriate therapy (5), the medical literature is scarce.

In the clinical setting of OGIB, the ESGE suggests using the Saurin's classification (P0-P1-P2, where P stands for “pertinence”) (6) for the evaluation of the clinical relevance of the lesions (7).

According to this classification (the publication includes a short list of examples), P0 lesions have no clinical relevance, P1 lesions have an uncertain hemorrhagic potential, and P2 lesions are highly relevant sources of bleeding.

Some clues are also given about the interpretation of inflammatory and ulcerative lesions in the Capsule Endoscopy Crohn's Disease Activity Index (CECDAI) (8) and in the Lewis score (9).

However, these scores aim to grade the severity of SBCE findings in patients with identified Crohn's disease, rather than to establish a diagnosis of Crohn's disease.

Therefore, the relevance of SBCE findings is based on a low level of knowledge.

The foregoing is not effective, and it would be desirable to improve the way of assessing the clinical relevance SBCE findings.

### SUMMARY OF INVENTION

Embodiments of the invention provide a method and a device for analyzing a video sequence captured by small bowel capsule endoscopy devices, as described in the accompanying claims.

Specific embodiments of the invention are set forth in the dependent claims.

These and other aspects of the invention will be apparent from and elucidated with reference to the embodiments described hereinafter.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Further details, aspects and embodiments of the proposed solution will be described, by way of example only, with reference to the drawings. In the drawings, like reference numbers are used to identify like or functionally similar elements. Elements in the figures are illustrated for simplicity and clarity and have not necessarily been drawn to scale.

FIG. 1 is a flow chart showing a method according to an embodiment of the invention.

FIG. 2 is a diagram showing a device according to an embodiment of the invention.

FIG. 3 is a screenshot showing an illustrated question of a questionnaire according to an embodiment of the invention.

FIG. 4 is a screenshot showing an illustrated finding according to an embodiment of the invention.

FIG. 5 shows Table 2 referred to below, which provides a key for interpreting data in subsequent tables.

FIG. 6 shows Table 3 referred to below, which presents results for findings related to Overt obscure gastrointestinal bleeding in accordance with methods herein described.

FIG. 7 shows Table 4 referred to below, which presents results for findings related to Occult obscure gastrointestinal bleeding in accordance with methods herein described.

FIG. 8 shows Table 5 referred to below, which presents results for findings related to Suspected Crohn's Disease in accordance with methods herein described.

### DESCRIPTION OF EMBODIMENTS

The invention relates to a method for analyzing a video sequence captured by Small Bowel (SB) Capsule Endoscopy (CE) devices, thereinafter referred to as SBCE devices.

In the invention, SBCE devices are placed in a patient's body, during a SBCE video examination, so as to detect abnormal findings, the clinical setting of the patient being chosen from suspected Crohn's Diseases (CD) or Obscure GastroIntestinal Bleeding (OGIB), in particular, overt OGIB and occult OGIB.

Furthermore, the method according to the invention analyzes the video sequence by calculating a level of “clinical relevance” of the findings detected by the SBCE devices according to the chosen clinical setting.

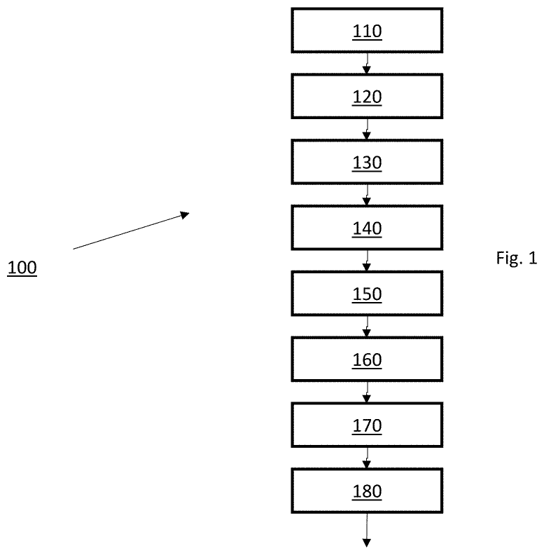

In FIG. 1, at step 110, the method 100 collects, by storing means, at least one video sequence captured during a SBCE video examination.

At step 120, the method 100 automatically detects, by calculating means, from the video sequence, at least one image comprising at least one abnormal detected finding in the Small Bowel. In the invention, the abnormal detected findings are selected from the group consisting of typical angiectasia (also known as angiodysplasia), red spot/red dot, erythematous patch, phlebectasia, diminutive angiectasia, aphthoid erosion, superficial ulceration, deep ulceration, edema, hyperemia, denudation, stenosis, lymphangiectasia, chylous cyst and blood.

According to the invention, the reader should readily understand that calculating means 220 connotes a specific structure.

Furthermore, the calculating means 220 may be a device that interprets and executes instructions, consisting of at least an instruction control unit and an arithmetic unit, such as, but not limited to, a central processing unit (CPU), a processor, or the like.

According to the invention, algorithms used by the calculating means 220 for detecting the abnormal detected findings include pure mathematical techniques and artificial intelligence techniques.

In an embodiment of step 120, at least one abnormal detected finding is confirmed by a human. Indeed, the method 100 may be used to only assist a human in detecting the abnormal findings observed in the Small Bowel. In that case, a detection made by the calculating means should only be considered as a proposed detection. In an example, the human confirmation is made before step 130.

At step 130, the method 100 counts, by the calculating means, the abnormal detected findings.

At step 140, the method 100 classifies, by the calculating means, the abnormal detected findings according to predetermined rules, further described in the “Materials and Methods”, “Discussion” and “Conclusion” sections below.

In particular, when at least one abnormal finding is detected among red spot/red dot, erythematous patch, phlebectasia, lymphangiectasia, chylous cyst, the method 100 classifies the abnormal detected finding as of “low clinical relevance”, this applying irrespective of the clinical setting.

Furthermore, when only one abnormal finding is detected among diminutive angiectasia, aphtoid erosion, hyperemia, denudation, the method 100 classifies the abnormal detected finding as of “low clinical relevance”, this applying irrespective of the clinical setting.

Further, when several abnormal findings are detected among aphthoid erosion, denudation, the method 100 classifies the abnormal detected findings as of “intermediate/doubtful clinical relevance” or “high clinical relevance”, according to the given clinical setting and to the number of abnormal detected findings.

Still further, when at least one abnormal finding is detected among superficial ulceration, deep ulceration, stenosis, and blood, the method 100 classifies the abnormal detected finding as of “intermediate/doubtful clinical relevance” or “high clinical relevance”, this applying to the given clinical setting and to the number of abnormal detected findings.

Finally, at step 150, the method 100 outputs, by displaying means, the “clinical relevance” of each abnormal detected finding.

According to the invention, the reader should readily understand that storing means 220 connotes a specific structure.

For example, the storing means 210 may be a device that stores data, such as, but not limited to, a memory, and integrated circuit memory, or the like.

Also, the displaying means 230 may be a text or a graphical display means, such as, but not limited to, a liquid crystal display (LCD), a plasma display, a light emitting diode (LED) display, a monitor, or the like.

In one embodiment of step 140, the method 100 further classifies, by the calculating means, the abnormal detected findings according to further predetermined rules, further described in the “Materials and Methods”, “Discussion” and “Conclusion” sections below.

In particular, when at least one abnormal finding is detected as blood, the method 100 classifies the abnormal detected finding as of “high clinical relevance”, this applying to the clinical setting of occult OGIB or overt OGIB.

Further, when at least one abnormal finding is detected among stenosis or deep ulceration, the method 100 classifies the abnormal detected finding as of “high clinical relevance”, this applying to the clinical setting of occult OGIB, overt OGIB or suspected CD.

In another embodiment of step 140, when at least one abnormal finding is detected among typical angiectasia (also known as angiodysplasia), deep ulceration, stenosis or blood, and/or when several abnormal findings are detected as superficial ulceration, the method 100 classifies the abnormal detected findings as of “high clinical relevance”, this applying to the clinical setting of overt OGIB.

In yet another embodiment of step 140, when several abnormal findings are detected among aphthoid erosion or denudation, and/or when only one abnormal finding is detected as superficial ulceration, the method 100 classifies the abnormal detected findings as of “intermediate/doubtful clinical relevance”, this applying to the clinical setting of overt OGIB.

In still another embodiment of step 140, when at least one abnormal finding is detected among diminutive angiectasia, edema, hyperemia, the method 100 classifies the abnormal detected findings as of “low clinical relevance”, this applying to the clinical setting of overt OGIB.

In another embodiment of step 140, when only one abnormal finding is detected as edema, the method 100 classifies the abnormal detected finding as of “low clinical relevance”, this applying to the clinical setting of occult OGIB.

In yet another embodiment of step 140, when several abnormal findings are detected among diminutive angiectasia, aphtoid erosion, edema, hyperemia, and/or when only one abnormal detected finding is detected among superficial ulceration, the method 100 classifies the abnormal detected findings as of “intermediate/doubtful clinical relevance”, this applying to the clinical setting of OGIB.

In still another embodiment of step 140, when at least one abnormal finding is detected among typical angiectasia (also known as angiodysplasia), deep ulceration, stenosis or blood, and/or several abnormal findings are detected as superficial ulceration, the method 100 classifies the abnormal detected finding as of “high clinical relevance”, this applying to the clinical setting of occult OGIB.

In another embodiment of step 140, when one or several abnormal findings are detected among typical angiectasia (also known as angiodysplasia), diminutive angiectasia, and/or less than six abnormal findings are detected as hyperemia, the method 100 classifies the abnormal detected findings as of “low clinical relevance”, this applying to the clinical setting of suspected CD.

In yet another embodiment of step 140, when only one abnormal finding is detected as superficial ulceration, and/or several abnormal finding are detected as denudation, and/or six or more abnormal findings are detected as hyperemia, and/or at least one abnormal finding is detected among edema or blood, the method 100 classifies the abnormal detected findings as of “intermediate/doubtful clinical relevance”, this applying to the clinical setting of suspected CD.

In still another embodiment of step 140, when several abnormal findings are detected among aphtoid erosion, superficial ulceration, and/or at least one abnormal finding is detected among deep ulceration, stenosis, the method 100 classifies the abnormal detected findings as of “high clinical relevance”, this applying to the clinical setting of suspected CD.

In an embodiment of step 110, the method 100 further comprises collecting all SBCE video examinations and detecting the abnormal findings for all the SBCE video examinations.

In another embodiment of the invention, the method 100 further comprises, at step 160, displaying on a user interface, by the displaying means, the outputs about the classification of the abnormal detected findings in terms of “low clinical relevance”, “intermediate/doubtful clinical relevance” and “high clinical relevance”.

In still another embodiment of the invention, the method 100 further comprises, at step 170, outputting, by the displaying means, an indication of the position of the abnormal detected finding within the image in which at least one abnormal finding is detected.

In yet another embodiment of the invention, the method 100 further comprises, at step 180, counting, by the calculating means, the number of abnormal detected findings which are classified as of “intermediate/doubtful clinical relevance” or “high clinical relevance”, this applying to the given clinical setting.

In another embodiment of the invention, the method 100 further comprises, at step 130, determining, by the calculating means, whether an abnormal detected finding appears on at least two different video sequences during the SBCE video examination.

The invention also relates to a device 200 for analyzing a video sequence captured by SBCE devices, when placed in a patient's body, the clinical setting of the patient being chosen from overt OGIB, occult OGIB or suspected CD.

Furthermore, the device 200 aims at calculating a level of “clinical relevance” of the findings detected by the SBCE devices according to the chosen clinical setting.

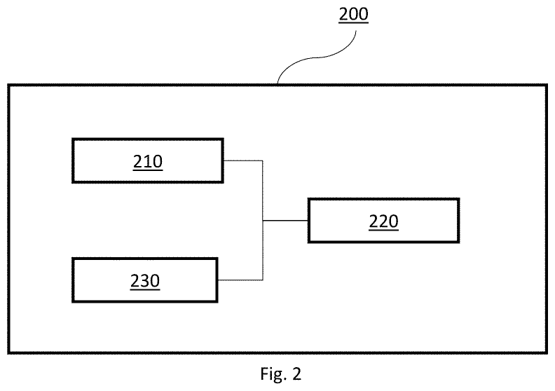

In FIG. 2, the device 200 comprises storing means 210, calculating means 220 and displaying means 230.

According to the invention, the reader should readily understand that storing means 210, calculating means 220 and displaying means 230, connote a specific structure.

For example, the storing means 210 may be a device that stores data, such as, but not limited to, a memory, and integrated circuit memory, or the like.

Furthermore, the calculating means 220 may be a device that interprets and executes instructions, consisting of at least an instruction control unit and an arithmetic unit, such as, but not limited to, a central processing unit (CPU), a processor, or the like.

Also, the displaying means 230 may be a text or a graphical display means, such as, but not limited to, a display, a liquid crystal display (LCD), a plasma display, a light emitting diode (LED) display, a monitor, or the like.

Storing means 210 are configured for collecting at least one set of a video sequence captured during a SBCE examination.

Calculating means 220 are configured for:
i) automatically detecting from the video sequence, at least one image comprising at least one abnormal finding in the Small Bowel detected among typical angiectasia (also known as angiodysplasia), red spot/red dot, erythematous patch, phlebectasia, diminutive angiectasia, aphthoid erosion, superficial ulceration, deep ulceration, edema, hyperemia, denudation, stenosis, lymphangiectasia, chylous cyst and blood;
ii) counting the abnormal detected findings;
iii) classifying the abnormal detected findings such that:
   when at least one abnormal finding is detected among red spot/red dot, erythematous patch, phlebectasia, lymphangiectasia, chylous cyst, then classifying the abnormal detected finding as of “low clinical relevance”, this applying irrespective of the clinical setting
   when only one abnormal finding is detected among diminutive angiectasia, aphtoid erosion, hyperemia, denudation, then classifying the abnormal detected finding as of “low clinical relevance”, this applying irrespective of the clinical setting,
   when several abnormal findings are detected among aphthoid erosion, denudation, then classifying the abnormal detected findings as of “intermediate/doubtful clinical relevance” or “high clinical relevance”, this applying to the chosen clinical setting and to the number of abnormal detected findings,
   when at least one lesion is detected among superficial ulceration, deep ulceration, stenosis, and blood, then classifying the abnormal detected finding as of “intermediate/doubtful clinical relevance” or “high clinical relevance”, this applying to the chosen clinical setting and to the number of abnormal detected findings detected.

Displaying means 230 are configured for outputting the “clinical relevance” of each abnormal detected finding.

In one embodiment, the calculating means further count the number of abnormal detected findings which are classified as of “intermediate/doubtful clinical relevance” or “high clinical relevance”, this applying to the chosen clinical setting.

In another embodiment, the calculating means further determine if the same abnormal detected finding appears on at least two different video sequences during the SBCE video examination.

In the foregoing specification, the proposed solution has been described with reference to specific examples of embodiments of the proposed solution. It will, however, be evident that various modifications and changes may be made therein without departing from the broader scope of the proposed solution as set forth in the appended claims.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of devices or logic blocks to achieve the same functionality is effectively “associated” such that the desired functionality is achieved. Hence, any two devices or logic blocks herein combined to achieve a particular functionality may be seen as “associated with” each other such that the desired functionality is achieved, irrespective of architectures or intermediate devices. Likewise, any two devices or logic blocks so associated can also be viewed as being “operably connected,” or “operably coupled,” to each other to achieve the desired functionality.

Furthermore, those skilled in the art will recognize that boundaries between the above-described operations are merely illustrative. The multiple operations may be combined into a single operation, a single operation may be distributed in additional operations and operations may be executed at least partially overlapping in time. Moreover, alternative embodiments may include multiple examples of a particular operation, and the order of operations may be altered in various other embodiments.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word “comprising” does not exclude the presence of other elements or operations than those listed in a claim. Furthermore, the terms “a” or “an,” as used herein, are defined as one or as more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

### Materials and Methods

### Study Design

The inventors designed a study design based on a series of illustrated script questions on the clinical relevance of CE findings in the SB, with various types and numbers of findings, in various clinical settings.

The questionnaire was prepared by a core group of three physicians, assisted by one premed student and one GI fellow. Sixteen types of SB findings were selected. The list included five vascular lesions (as listed and defined by a previous consensus (3)): “typical angiectasia”, “red spot/dot”, “erythematous patch”, “phlebectasia”, “diminutive angiectasia”); seven inflammatory and ulcerative lesions (as listed and defined by another previous consensus (4)): “aphthoid erosion”, “superficial ulceration”, “deep ulceration”, “edema”, “denudation”, “hyperemia”, “stenosis”); two lymphatic lesions (“chylous cysts”, “lymphangiectasia”); “blood”; and normal frames (used as controls).

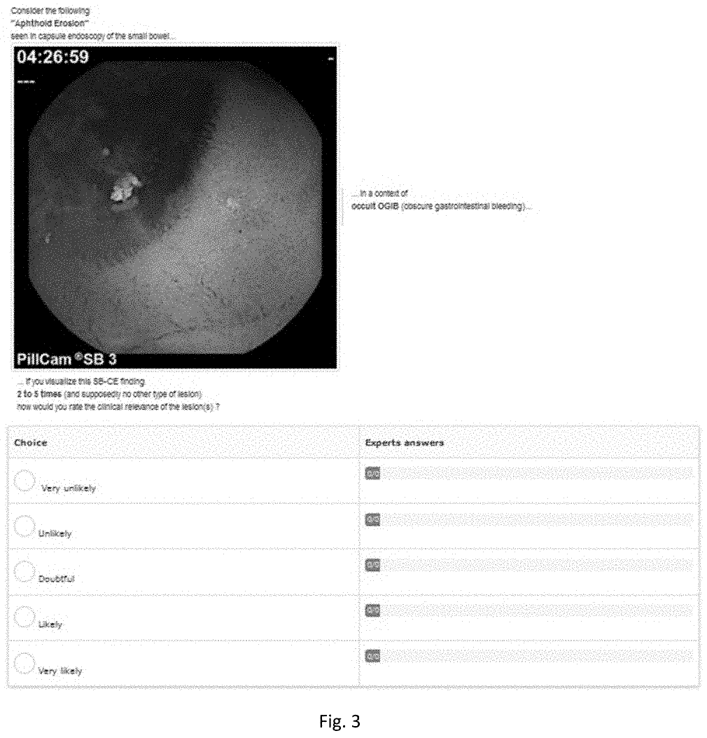

FIG. 3 shows a screenshot of an internet-based questionnaire, displaying an illustrated question, with one type of finding, one category of numbers, and one clinical setting.

Hence, according to FIG. 3, each question displayed a third-generation SBCE still frame associated with a written diagnosis. Four different illustrations were chosen for each of the sixteen types of SB findings. All illustrated findings included for voting were labeled with a precise diagnosis. Tumors, polyps, and coeliac disease were not addressed in this study.

For each illustrated finding, a group of experts had to provide their interpretation in three circumstances (this is later referred to as number category):
if the finding identified in the SBCE was observed only once (a single lesion),
if the finding was observed 2 to 5 times, and
if the finding was found six or more times.

In particular, the experts were asked to give their opinion on the relevance (for diagnosis and/or for start or change of a therapy) of this type and number of finding(s) in the three following clinical settings: overt OGIB/occult OGIB/suspected Crohn's disease.

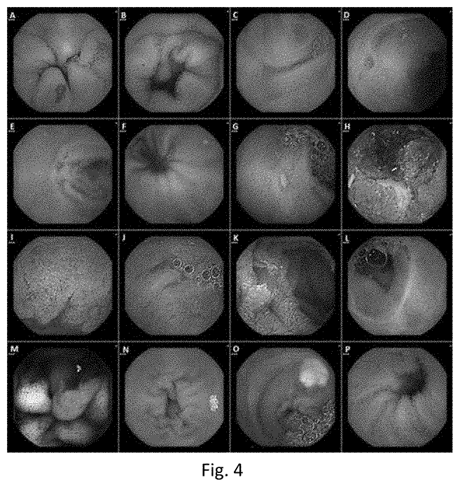

FIG. 4 shows examples of illustrations of the different types of findings assessed for their clinical relevance on the internet-based questionnaire. In FIG. 4, (A) shows a typical angiectasia; (B) shows a red dot/spot; (C) shows an erythematous patch; (D) shows a phlebectasia; (E) shows a diminutive angiectasia; (F) shows an aphthoid erosion; (G) shows a superficial ulceration; (H) shows a deep ulceration; (I) shows an edema; (J) shows an hyperemia; (K) shows a denudation; (L) shows a stenosis; (M) shows blood; (N) shows a lymphangiectasia; (O) shows a chylous cyst; (P) shows a normal mucosa; (3,4).

Hence, as shown in Table 1 and according to FIG. 4, the experts were asked to rate the clinical relevance of the labeled and illustrated findings for each specific question, by taking into account its type and number category, and the clinical setting, as follows: very unlikely (−2)/unlikely (−1)/doubtful (0)/likely (+1)/very likely (+2).

TABLE 1
Numerical scale for voting process

| Vote | Numerical value |
|---|---|
| Very unlikely | −2 |
| Unlikely | −1 |
| Doubtful | 0 |
| Likely | 1 |
| Very likely | 2 |

Overall, through this questionnaire, each individual expert had to answer a set of four different labeled and illustrated questions (named “scenario”) for one type of finding (among the sixteen types of SB findings), one number category (among the three different categories), and one clinical setting (among the three different ones), which makes a total of one hundred ninety-two scenarios, with a total of five hundred seventy-six illustrated questions per expert.

This approach (four questions per scenario) was chosen first because the answers of the experts to the very same questions may not be perfectly reproducible, and second because the expert's opinion may vary with the different illustrations of a specific type of finding.

The questionnaire was internet-based using an e-learning electronic platform. The five hundred seventy-six questions were shuffled. The core group checked that the four questions within a scenario were never consecutive. Furthermore, the experts were allowed to log in and out at any time until they completed all five hundred seventy-six questions.

### Expert Group

According to the methodology of script questionnaires, the core group decided to include ten to twenty experts in the study (10,11). An expert in the field of SBCE was defined as a board-certified gastroenterologist with current clinical, teaching and research activities in SBCE. A minimum of five years' experience in SBCE reading, with annual reading of at least fifty video recordings a year, teaching activities, and publications related to SBCE in international peer-reviewed journals, were required to be considered an expert. Fifteen international experts fulfilling these criteria were invited.

Analysis

All answers were reviewed and analyzed. A mean score was first calculated per expert and per scenario. The median score (with first Q1 and third Q3 quartiles) of the fourteen mean scores per scenario obtained from fourteen experts were then calculated. The clinical relevance of findings was based on these median scores per scenario.

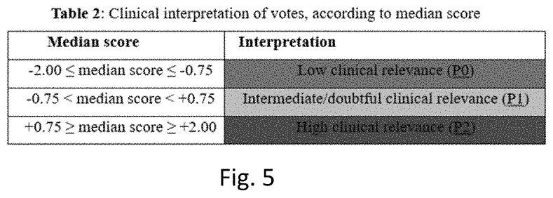

Table 2 (shown at FIG. 5) provides the keys for interpretation of the median scores. In particular, findings with a median score below or equal to −0.75 were considered to have a low clinical relevance (P0). Findings with a median score between −0.75 and +0.75 were considered to have an intermediate/doubtful clinical relevance (P1), whereas those with a mean score over +0.75 were considered to have a high clinical relevance (P2). Other quantitative data were reported as mean±standard deviation (SD).

### Results

Fourteen experts participated to the study, and one declined the invitation (participation rate 93.3%). The fourteen participating experts were based in France (n=2), Germany (n=1), Ireland (n=1), Israel (n=1), Italy (n=2), Portugal (n=1), Spain (n=1), Sweden (n=1) and United Kingdom (n=4).

The mean age of the members of the experts' group was 47.6±9.7 years. The mean CE reading experience of these experts was 15.5±2.9 years year, with a mean declared number of 200±126 readings annually.

All were active SBCE readers and were trainers for SBCE reading.

All were authors of publications related to SBCE in international peer-reviewed journals.

The fourteen participating experts completed all five hundred seventy-six questions of the questionnaire (completion rate 100%): eight thousand sixty-four answers were therefore available for analysis.

Normal frames served as controls, and had median score of −2.00, whatever the numbers of findings and the clinical settings.

Lymphatic findings (“lymphangiectasia” and “chylous cyst”) were also consistently rated of low pertinence (P0), with median score of −2.00 in all scenarios.

### Results for Overt Obscure Gastrointestinal Bleeding

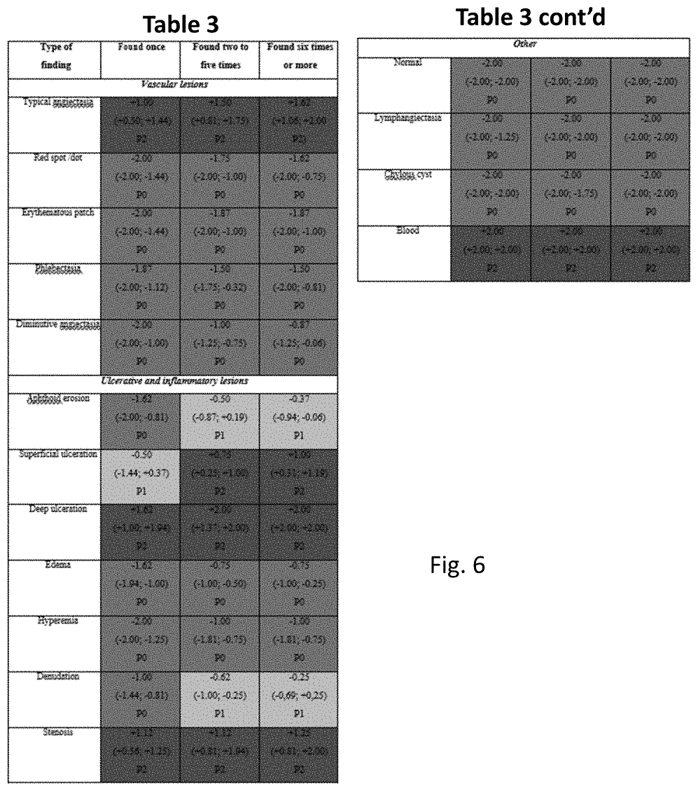

As shown in Table 3 (see FIG. 6), any finding of “typical angiectasia”, “deep ulceration”, “stenosis”, “blood”, and multiple findings of “superficial ulcerations”, were considered highly relevant (P2) in the clinical setting of overt OGIB. Findings of isolated “superficial ulceration” and of multiple “aphthoid erosion” and “denudation” were of intermediate/doubtful pertinence (P1). Other scenarios had low relevance median scores (P0) in this clinical setting.

Table 3, shown in FIG. 6, sets forth the interpretation of the votes in the clinical setting of overt obscure gastrointestinal bleeding. Each box in Table 3 displays the median (Q1; Q3) score of the mean scores obtained from the fourteen experts to four different illustrated questions (fifty-six answers per “scenario”). P0, P1 or P2 interpretations are determined according to Table 2 (shown in FIG. 5).

### Results for Occult Obscure Gastrointestinal Bleeding

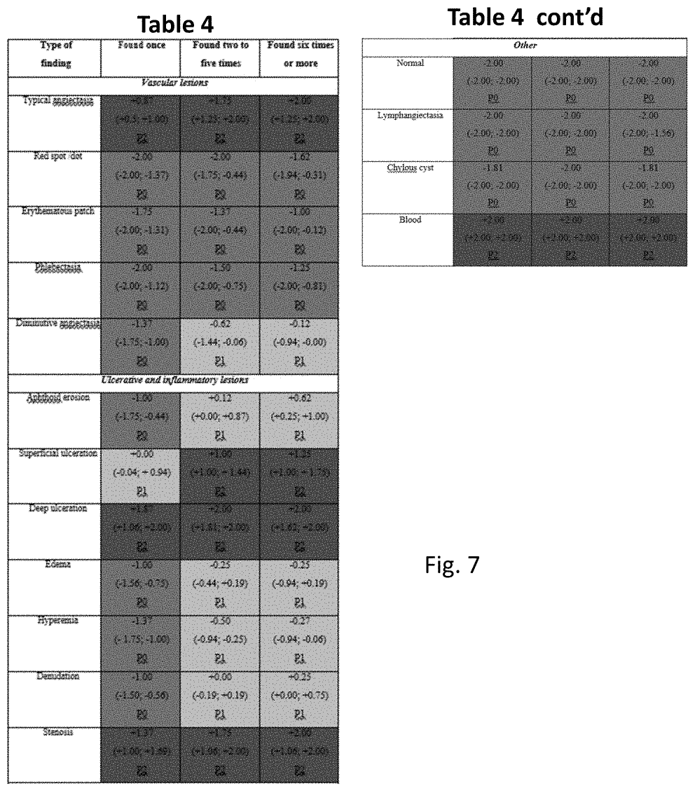

Table 4, shown in FIG. 7, shows details regarding the results in this clinical setting. Overall, “typical angiectasia”, “deep ulceration”, “stenosis” and “blood” were categorized as highly relevant (P2), whatever their numbers. “Superficial ulceration” was considered of high clinical relevance (P2) when seen at multiple sites, but of intermediate/doubtful relevance (P1) when found only once. Findings of “diminutive angiectasia”, “aphthoid erosion”, “edema”, “hyperemia” and “denudation”, were of intermediate/doubtful relevance (P1) when found at multiple sites, and of low relevance (P0) when only found once. Other scenarios had low relevance median scores (P0) in this clinical setting. In Table 4 (FIG. 7), interpretation of the votes, in the clinical setting of occult obscure gastrointestinal bleeding, is presented. Each box in Table 4 displays the median (Q1; Q3) score of the mean scores obtained from the fourteen experts to four different illustrated questions (fifty-six answers per “scenario”). P0, P1 or P2 interpretations are determined according to Table 2 (shown in FIG. 5).

### Results for Suspected Crohn's Disease

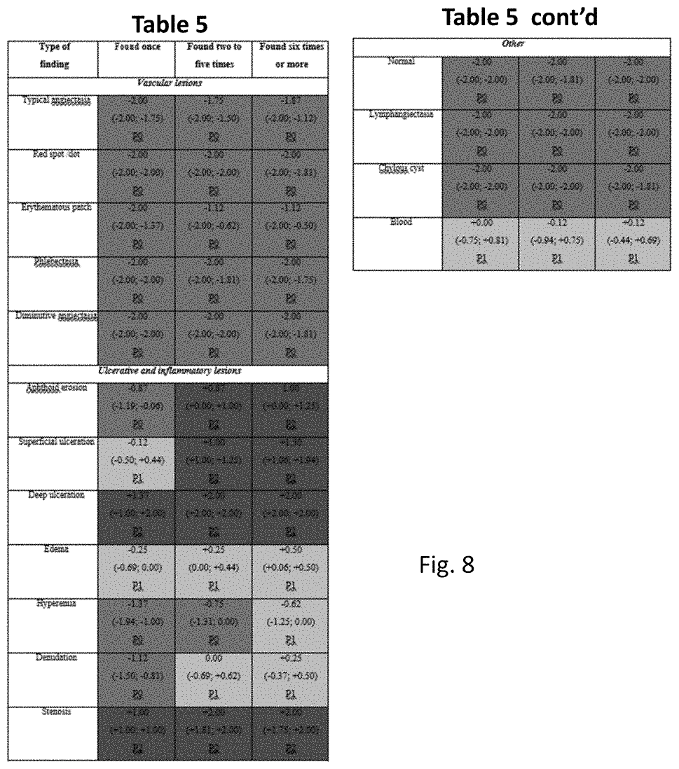

As shown in Table 5, which is shown in FIG. 8, any finding of “deep ulceration” or “stenosis”, and multiple findings of “superficial ulceration” and “aphthoid erosion”, were considered highly relevant (P2) in this clinical setting. An isolated finding of “superficial ulceration” was deemed of intermediate/doubtful relevance (P1) and a finding of “aphthoid erosion” was deemed of low relevance (P0). Findings of “edema”, “hyperemia” and “denudation” were of low to intermediate/doubtful relevance, according to their number category. A finding of “blood” was of intermediate/doubtful relevance (P1), with large interquartile ranges (IQR). All vascular lesions (“typical angiectasia”, “diminutive angiectasia”, “red spot/dot”, “erythematous patch”, “phlebectasia”) were considered of low clinical relevance (P0).

In Table 5 (FIG. 8), interpretation of the votes, in the clinical setting of suspected Crohn's disease, is presented. Each box in Table 5 displays the median (Q1; Q3) score of the mean scores obtained from the fourteen experts to four different illustrated questions (fifty-six answers per “scenario”). P0, P1 or P2 interpretations are determined according to Table 2 (shown in FIG. 5).

### Discussion

### Study Design

The study provides a stronger basis for the definition of three different levels of clinical relevance (P0, P1, and P2), as proposed by Saurin et al. (6), regarding various numbers/ranges of sixteen types of findings, in the three most frequent clinical settings for which a SBCE examination is recommended. These results are based on the analysis of eight thousand sixty-four answers of fourteen international experts to an illustrated script questionnaire.

Overall, this study suggests that findings of “red spots/dot”, “erythematous patch”, “phlebectasia”, “lymphangiectasia” and “chylous cysts” are of low clinical relevance (P0), and may be considered as normal variants in most cases, as recently proposed by other authors (12). Conversely, findings of “deep ulceration” or “stenosis” are highly relevant (P2) in most cases. The interpretation of other findings is more subtle and may vary according to the clinical setting and to the number of similar findings in one recording.

The results of the experts to the script questionnaire in the clinical settings of overt and occult OGIB are given with details in Table 3 and Table 4 and can be discussed in a more global manner.

Findings of “typical angiectasias” were clinically relevant to the experts in all scenarios. In the original proposal by Saurin et al. (6), “angiectasias” were listed as findings with a high hemorrhagic potential (P2). In the 1-year follow-up of the patients of this series, 61% of those with P2 lesions were treated, compared to 23% of patients with P1 or P0 lesions (13). These results are consistent with those of a prospective, multicenter study, where patients undergoing an endoscopic ablation of “typical angiectasia” (P2) had a significantly lower risk of bleeding recurrence than patients with intermediate/doubtful pertinence (P1) SB vascular lesions (14). “Diminutive angiectasias” were deemed to be of intermediate/doubtful clinical relevance (P1) when multiple (and P0 when singular) in scenarios of occult OGIB. Any other of the listed vascular lesions were of low relevance (P0), whatever their numbers and the clinical setting. All other vascular lesions were categorized P0 lesions, therefore deemed unlikely to be responsible for OGIB according to the experts.

Overall, inflammatory and ulcerative lesions in the clinical setting of OGIB, whether overt or patent, were considered of interest by the experts in terms of clinical relevance, with higher median scores with deeper tissue loss (from “denudation” to “deep ulceration”), and when found at multiple sites. The presence of “blood” was always deemed of high relevance (P2, with a median score of +2.0 in all cases) in the clinical setting of OGIB, and this can be seen as a positive control for the inventor's study. In some studies that focus on SBCE examination for OGIB, a specific “P3’ score for ‘blood’ (14) was extended from the original score by Saurin et al. that had only three categories (P0, P1, and P2) (6).

In the clinical setting of suspected Crohn's disease, a global interpretation of the results is possible as well.

Unsurprisingly, none of the listed vascular lesions was deemed of interest (all had a median score below −1.0, with a P0 interpretation) whatever their type and number. Conversely, all inflammatory or ulcerative lesions were relevant to the diagnosis of Crohn's disease, with a stepwise increase in median score with higher numbers of findings, and with deeper tissue loss (from ‘denudation’ to ‘aphthoid erosion’, and then to ‘superficial’ and ‘deep’ ulcerations). Still, stigmata of inflammation and ulceration are not specific signs of Crohn's disease, and the interpretation regarding their clinical relevance was often intermediate/doubtful (P1), as illustrated by the lower median scores of more subtle lesions (‘hyperemia’, ‘denudation’, ‘edema’, and isolated ‘aphthoid erosion’) compared to ‘ulcerations’ (whether superficial or deep). Findings of ‘stenosis’ were probably considered more specific of Crohn's disease, whether isolated or multiple, and were therefore quite consistently categorized as highly relevant findings (P2) by experts.

Overall, the inventor's global reading of the results is consistent with both Lewis score (5) and CECDAI (4), where ‘stenosis’/‘stricture’ is a specific item, and where the extent/width of tissue inflammation or tissue loss increases both scores. Similarly, in the original classification by Saurin et al., a “large ulceration” was considered highly relevant (P2) in terms of bleeding potential (6). Interestingly, the interpretation of the presence of “blood” was inconsistent for experts in cases of suspected Crohn's disease, with a median score equal or close to 0.0, but with an IQR over 1.0.

Up to now, any evidence on the relevance of lesions found in SBCE was weak. Most SBCE reports, courses and outcome measurements for clinical studies relied on the judgment, insight, opinion and experience of the physician/teacher/investigator.

A script questionnaire is considered one of the most reliable and valid tools for the assessment of judgment and clinical reasoning (15-17). A script questionnaire was more appropriate to build up a guide than a Delphi process that aims to draw a consensus.

This study has several strengths regarding this methodology.

First, a large number of well-selected experts were involved, as proposed in the standard methodology for script questionnaires (10,11,18).

Second, the questionnaires were illustrated with high-quality, third-generation SBCE images, with vascular and ulcerative/inflammatory findings being named and described according to two international Delphi consensus (3,4).

Third, the high (five hundred seventy-six) number of questions proposed to the experts, in a random (non-consecutive) order, provided a great overview of their clinical thinking regarding various types and numbers of findings, in different clinical settings, named “scenarios”.

Fourth, the internet-based questionnaire allowed high participation (93%) and completion (100%) rates, and independent answers from the experts.

Fifth, normal images were also interpreted, thus providing a control group of images, where median pertinence scores were consistently at the lowest level (−2.00). Similarly, “blood” was a positive control in the clinical setting of OGIB, with scores consistently among at the highest level (+2.00).

These results in both control groups strengthen the internal validity of the study.

The study also has some limitations.

First, the list of 16 SB findings included for voting was established by a core group of non-voting investigators, based on their experience and on previous Delphi consensus (3,4) which could have been extended. Noticeably, the core group decided not to include obvious relevant lesions to the experts (mainly protruding lesions, such as polyps, tumors, varices, and submucosal lesions).

Second, the level of evidence employed remains low, although higher than that of a single expert's opinion (as seen today in reports, in courses, and in study protocols). Longitudinal studies could bring higher levels of evidence, but their feasibility is challenging (13).

Last, the clinical settings/scenarios were restricted to three basic indications, but SBCE examination actually covers a wider range of clinical situations in routine practice.

The results presented here aim to better standardize SBCE reading, reporting, teaching and research.

Indeed, in a previous study by Saurin et al., a tandem reading of first-generation SBCE recordings showed a 60.4% interobserver concordance rate regarding the pertinence of two hundred twenty-five findings in sixty patients with OGIB (6). Concordance rates were 59.1%, 51.2% and 75.9% for P0, P1 and P2 lesions, respectively.

The CECDAI and the Lewis scores are validated to grade the severity of SBCE findings in patients with known Crohn's disease (8,9), but they have not been evaluated as diagnostic tools. The inventors therefore believe that the inventor's results bring a higher level of knowledge regarding the clinical relevance of SB findings in the main indications for CE. Still, many median and IQR scores in the inventor's guide reflect some uncertainty from the experts regarding the relevance of subtle findings seen in SBCE. Indeed, most P1 findings should be considered with caution (not overinterpreting and not overlooking . . . ), as well as some P0 and P2 findings where the IQR is broad and/or overlapping −0.75 and +0.75 thresholds.

Moreover, when talking about relevance, it is important to integrate as much clinical information as possible. For example, OGIB was divided in the questionnaire as overt or occult, but the interpretation of SBCE may vary a lot, depending on other clinical factors. Active ongoing vs. past bleeding, the age, gender, comorbidities, treatments (antiplatelet agents, anticoagulants, non-steroidal anti-inflammatory drugs) of patients and subtle findings (Helicobacter pylori gastritis, diverticulosis) as well as quality indicators (completeness and cleanliness of bidirectional endoscopy for instance) can all affect interpretation.

Similarly, findings deemed herein of intermediate/doubtful relevance SBCE in suspected Crohn's disease may be interpreted differently with the availability of additional data such as symptoms, treatments (most importantly intake of non-steroidal anti-inflammatory drugs), laboratory work up, imaging, and pathology reports.

### CONCLUSION

SBCE has become a routine endoscopy examination. Efforts have been made to standardize CE training courses, with an international core curriculum having been created (19). The assessment of SBCE reading competencies is still to be defined and classified in many countries (20,21).

To date, terminology, description of CE findings (3,4), and standards for quality reporting (22) for SBCE have been published, all of which aid in developing a CE reporting consensus.

The ESGE recommends grading the level of relevance of SBCE using scores (7). The inventors believe that this study will help standardize SBCE interpretation and reporting by physicians, and possibly by automated, artificial intelligence-based systems

The inventors believe that the proposed new standards (names, descriptions, and levels of clinical relevance), based on surveys designed by a core group and addressed to international experts, will also help to better define outcome measurements for research study protocols.

### BIBLIOGRAPHIC REFERENCES

(1) Iddan G, Meron G, Glukhovsky A, Swain P. Wireless capsule endoscopy. Nature. 2000; 405(6785):417.
(2) Pennazio M, Spada C, Eliakim R, Keuchel M, May A, Mulder C J, et al. Small-bowel capsule endoscopy and device-assisted enteroscopy for diagnosis and treatment of small-bowel disorders: European Society of Gastrointestinal Endoscopy (ESGE) Clinical Guideline. Endoscopy. 2015; 47(4):352-76.
(3) Leenhardt R, Li C, Koulaouzidis A, Cavallaro F, Cholet F, Eliakim R, et al. Nomenclature and semantic description of vascular lesions in small bowel capsule endoscopy: an international Delphi consensus statement. Endosc Int Open. 2019; 7(3):E372-9.
(4) Leenhardt R, Buisson A, Bourreille A, Marteau P, Koulaouzidis A, Li C, et al. Nomenclature and semantic descriptions of ulcerative and inflammatory lesions seen in Crohn's disease in small bowel capsule endoscopy: An international Delphi consensus statement. United Eur Gastroenterol J. 2020 [Epub ahead of print]
(5) Saurin J C, Pioche M. Why should we systematically specify the clinical relevance of images observed at capsule endoscopy? Endosc Int Open. 2014; 2(2):E88-89.
(6) Saurin J-C, Delvaux M, Gaudin J-L, Fassler I, Villarejo J, Vahedi K, et al. Diagnostic value of endoscopic capsule in patients with obscure digestive bleeding: blinded comparison with video push-enteroscopy. Endoscopy. 2003; 35(7):576-84.
(7) Spada C, McNamara D, Despott E J, Adler S, Cash B D, Fernandez-Urién I, et al. Performance measures for small-bowel endoscopy: A European Society of Gastrointestinal Endoscopy (ESGE) Quality Improvement Initiative. United Eur Gastroenterol J. 2019; 7(5):614-41.
(8) Gal E, Geller A, Fraser G, Levi Z, Niv Y. Assessment and validation of the new capsule endoscopy Crohn's disease activity index (CECDAI). Dig Dis Sci. 2008; 53(7):1933-7.
(9) Gralnek I M, Defranchis R, Seidman E, Leighton J A, Legnani P, Lewis B S. Development of a capsule endoscopy scoring index for small bowel mucosal inflammatory change. Aliment Pharmacol Ther. 2008; 27(2):146-54.
(10) Gagnon R, Charlin B, Coletti M, Sauvé E, van der Vleuten C. Assessment in the context of uncertainty: how many members are needed on the panel of reference of a script concordance test? Med Educ. 2005; 39(3):284-91.
(11) Lubarsky S, Dory V, Duggan P, Gagnon R, Charlin B. Script concordance testing: from theory to practice: AMEE guide no. 75. Med Teach. 2013; 35(3):184-93.
(12) Ding Z, Shi H, Zhang H, Meng L, Fan M, Han C, et al. Gastroenterologist-level Identification of Small Bowel Diseases and Normal Variants by Capsule Endoscopy Using a Deep-learning Model. Gastroenterology. 2019; 157(4):1044-1054
(13) Saurin J C, Delvaux M, Vahedi K, Gaudin J L, Villarejo J, Florent C, et al. Clinical impact of capsule endoscopy compared to push enteroscopy: 1-year follow-up study. Endoscopy. 2005; 37(4):318-23.
(14) Rahmi G, Samaha E, Vahedi K, Delvaux M, Gay G, Lamouliatte H, et al. Long-term follow-up of patients undergoing capsule and double-balloon enteroscopy for identification and treatment of small-bowel vascular lesions: a prospective, multicenter study. Endoscopy. 2014; 46(7):591-7.
(15) Karila L, Francois H, Monnet X, Noel N, Roupret M, Gajdos V, et al. [The Script Concordance Test: A multimodal teaching tool]. Rev Med Interne. 2018; 39(7):566-73.
(16) Lubarsky S, Charlin B, Cook D A, Chalk C, van der Vleuten C P M. Script concordance testing: a review of published validity evidence. Med Educ. 2011; 45(4):329-38.
(17) Nazim S M, Talati J J, Pinjani S, Biyabani S R, Ather M H, Norcini J J. Assessing clinical reasoning skills using Script Concordance Test (SCT) and extended matching questions (EMQs): A pilot for urology trainees. J Adv Med Educ Prof 2019; 7(1):7-13.
(18) Lubarsky S, Gagnon R, Charlin B. Scoring the Script Concordance Test: not a black and white issue. Med Educ. 2013; 47(12):1159-61.
(19) Fernandez-Urien I, Panter S, Carretero C, Davison C, Dray X, Fedorov E, et al. International core curriculum for capsule endoscopy training courses. Endosc Int Open. 2017; 5(6):E526-38.
(20) Rajan E, Iyer P G, Oxentenko A S, Pardi D S, Alexander J A, Baron T H, et al. Training in small-bowel capsule endoscopy: assessing and defining competency. Gastrointest Endosc. 2013; 78(4):617-22.
(21) Rajan E, Martinez M, Gorospe E, Al Bawardy B, Dobashi A, Mara K C, et al. Prospective Multicenter Study to Evaluate Capsule Endoscopy Competency using a Validated Assessment Tool. Gastrointest Endosc. 2019 [Epub ahead of print]
(22) Korman L Y, Delvaux M, Gay G, Hagenmuller F, Keuchel M, Friedman S, et al. Capsule endoscopy structured terminology (CEST): proposal of a standardized and structured terminology for reporting capsule endoscopy procedures. Endoscopy. 2005; 37(10):951-9.

What is claimed is:

1. A method for analyzing a video sequence captured by Small Bowel (SB) Capsule Endoscopy (CE) devices, when placed in a patient's body during a SBCE video examination, a clinical setting of the patient being chosen from overt Obscure GastroIntestinal Bleeding (OGIB), occult OGIB, or suspected Crohn's Diseases (CD), by calculating a level of “clinical relevance” of the findings detected by the SBCE devices according to the chosen clinical setting, comprising:
(a) collecting, by a memory, at least one video sequence captured during a SBCE video examination;
(b) automatically detecting, by a processor, from the video sequence, at least one image comprising at least one abnormal finding in the Small Bowel detected among typical angiectasia, red spot/red dot, erythematous patch, phlebectasia, diminutive angiectasia, aphthoid erosion, superficial ulceration, deep ulceration, edema, hyperemia, denudation, stenosis, lymphangiectasia, chylous cyst and blood;
(c) counting, by the processor, the abnormal detected findings;
(d) classifying, by the processor, the abnormal detected findings such that:
i) when at least one abnormal finding is detected among red spot/red dot, erythematous patch, phlebectasia, lymphangiectasia, chylous cyst, then classifying the abnormal detected finding as of “low clinical relevance”, this applying irrespective of the clinical setting,
ii) when only one abnormal finding is detected among diminutive angiectasia, aphtoid erosion, hyperemia, denudation, then classifying the abnormal detected finding as of “low clinical relevance”, this applying irrespective of the clinical setting,
iii) when several abnormal findings are detected among aphthoid erosion, denudation, then classifying the abnormal detected findings as of “intermediate/doubtful clinical relevance” or “high clinical relevance”, this applying to the chosen clinical setting and to the number of abnormal detected findings,
iv) when at least one abnormal finding is detected among superficial ulceration, deep ulceration, stenosis, and blood, then classifying the abnormal detected finding as of “intermediate/doubtful clinical relevance” or “high clinical relevance,” this applying to the chosen clinical setting and to the number of abnormal detected findings;
(e) outputting, by a display, the “clinical relevance” of each abnormal detected finding.

2. The method according to claim 1, wherein in step (b), at least one abnormal detected finding is confirmed by a human.

3. The method according to claim 1, wherein in step (b),
when at least one abnormal finding is detected as blood, then classifying the abnormal detected finding as of “high clinical relevance”, this applying to the clinical setting of occult OGIB or overt OGIB; and,
when at least one abnormal finding is detected among stenosis or deep ulceration, then classifying the detected finding as of “high clinical relevance”, this applying to the clinical setting of occult OGIB, overt OGIB or suspected CD.

4. The method according to claim 1, wherein in step (b), when at least one abnormal finding is detected among typical angiectasia, deep ulceration, stenosis or blood, and/or when several abnormal findings are detected as superficial ulceration, then classifying the abnormal detected findings as of “high clinical relevance”, this applying to the clinical setting of overt OGIB.

5. The method according to claim 1, wherein in step (b), when several abnormal findings are detected among aphthoid erosion or denudation, and/or when only one abnormal finding is detected as superficial ulceration, then classifying the abnormal detected findings as of “intermediate/doubtful clinical relevance”, this applying to the clinical setting of overt OGIB.

6. The method according to claim 1, wherein in step (b), when at least one abnormal finding is detected among diminutive angiectasia, edema, hyperemia, then classifying the abnormal detected findings as of “low clinical relevance”, this applying to the clinical setting of overt OGIB.

7. The method according to claim 1, wherein in step (b), when only one abnormal finding is detected as edema, then classifying the abnormal detected finding as of “low clinical relevance”, this applying to the clinical setting of occult OGIB.

8. The method according to claim 1, wherein in step (b), when several abnormal findings are detected among diminutive angiectasia, aphtoid erosion, edema, hyperemia, and/or when only one abnormal detected finding is detected among superficial ulceration, then classifying the abnormal detected findings as of “intermediate/doubtful clinical relevance”, this applying to the clinical setting of OGIB.

9. The method according to claim 1, wherein in step (b), when at least one abnormal finding is detected among typical angiectasia, deep ulceration, stenosis or blood, and/or several abnormal findings are detected as superficial ulceration, then classifying the abnormal detected finding as of “high clinical relevance”, this applying to the clinical setting of occult OGIB.

10. The method according to claim 1, wherein when one or several abnormal findings are detected among typical angiectasia, diminutive angiectasia, and/or less than six abnormal findings are detected as hyperemia, then classifying the abnormal detected findings as of “low clinical relevance”, this applying to the clinical setting of suspected CD.

11. The method according to claim 1, wherein in step (b), when only one abnormal finding is detected as superficial ulceration, and/or several abnormal finding are detected as denudation, and/or six or more abnormal findings are detected as hyperemia, and/or at least one abnormal finding is detected among edema or blood, then classifying the abnormal detected findings as of “intermediate/doubtful clinical relevance”, this applying to the clinical setting of suspected CD.

12. The method according to claim 1, wherein in step (b), when several abnormal findings are detected among aphtoid erosion, superficial ulceration, and/or at least one abnormal finding is detected among deep ulceration, stenosis, then classifying the abnormal detected findings as of “high clinical relevance”, this applying to the clinical setting of suspected CD.

13. The method according to any of claim 1 step (a) further comprising collecting all SBCE video examinations and detecting the abnormal findings for all the SBCE video examinations.

14. The method according to any of claim 1, further comprising displaying on a user interface, by the display, the outputs about the classification of the abnormal detected findings in terms of “low clinical relevance”, “intermediate/doubtful clinical relevance” and “high clinical relevance”.

15. The method according to any of claim 1, further comprising outputting, by the display, an indication of the position of the abnormal detected finding within the image in which at least one abnormal finding is detected.

16. The method according to claim 1, further comprising a step of counting, by the processor, the number of abnormal detected findings which are classified as of “intermediate/doubtful clinical relevance” or “high clinical relevance”, this applying to the chosen clinical setting.

17. The method according to any of claim 1, step (c) further comprising determining, by the processor, when an abnormal detected finding appears on at least two different video sequences during the SBCE video examination.

18. A device for analyzing a video sequence captured by Small Bowel (SB) Capsule Endoscopy (CE) devices, when placed in a patient's body during a SBCE video examination, a clinical setting of the patient being chosen from overt Obscure GastroIntestinal Bleeding (OGIB), occult OGIB, or suspected Crohn's Diseases CD, by calculating a level of “clinical relevance” of the findings detected by the SBCE devices according to the chosen clinical setting, the device comprising:
a memory for collecting at least one set of a video sequence captured during a SBCE examination; and,
a processor configured for:
i) automatically detecting from the video sequence, at least one image comprising at least one abnormal finding in the Small Bowel detected among typical angiectasia, red spot/red dot, erythematous patch, phlebectasia, diminutive angiectasia, aphthoid erosion, superficial ulceration, deep ulceration, edema, hyperemia, denudation, stenosis, lymphangiectasia, chylous cyst and blood;
ii) counting the abnormal detected findings;
iii) classifying the abnormal detected findings such that:
when at least one abnormal finding is detected among red spot/red dot, erythematous patch, phlebectasia, lymphangiectasia, chylous cyst, then classifying the abnormal detected finding as of “low clinical relevance”, this applying irrespective of the clinical setting,
when only one abnormal finding is detected among diminutive angiectasia, aphtoid erosion, hyperemia, denudation, then classifying the abnormal detected finding as of “low clinical relevance”, this applying irrespective of the clinical setting,
when several abnormal findings are detected among aphthoid erosion, denudation, then classifying the abnormal detected findings as of “intermediate/doubtful clinical relevance” or “high clinical relevance”, this applying to the chosen clinical setting and to the number of abnormal detected findings,
when at least one lesion is detected among superficial ulceration, deep ulceration, stenosis, and blood, then classifying the abnormal detected finding as of “intermediate/doubtful clinical relevance” or “high clinical relevance”, this applying to the chosen clinical setting and to the number of abnormal detected findings detected;
iv) outputting, by a display, the “clinical relevance” of each abnormal detected finding.

19. The device according to claim 18, wherein the processor is further configured to count the number of abnormal detected findings which are classified as of “intermediate/doubtful clinical relevance” or “high clinical relevance”, this applying to the chosen clinical setting.

20. The device according to claim 18, wherein the processor is further configured to determine whether an abnormal detected finding appears on at least two different video sequences during the SBCE video examination.
